(19) United States
(12) Patent Application Publication
(10) Pub. No.: US 2021/0106252 A1
(43) Pub. Date: Apr. 15, 2021

(54) MAGNETIC RESONANCE IMAGING APPARATUS AND IMAGE PROCESSING APPARATUS

(71) Applicant: CANON MEDICAL SYSTEMS CORPORATION, Otawara-shi (JP)

(72) Inventors: Fumiyasu SHINKAI, Yaita (JP); Motohisa YOKOI, Nasushiobara (JP); Shinichi UCHIZONO, Nasushiobara (JP); Kazuhiro SUEOKA, Otawara (JP); Yutaka MACHII, Nasushiobara (JP)

(73) Assignee: CANON MEDICAL SYSTEMS CORPORATION, Otawara-shi (JP)

(21) Appl. No.: 17/060,753

(22) Filed: Oct. 1, 2020

(30) Foreign Application Priority Data
Oct. 9, 2019 (JP) 2019-186184

(51) Int. Cl. A61B 5/055; G01R 33/50; G01R 33/56

(52) U.S. Cl. CPC A61B 5/055; G01R 33/5602; G01R 33/50

(57) ABSTRACT

A magnetic resonance imaging apparatus according to an embodiment includes a processor. The processor acquires age information representing the length of time elapsed from the birth of a subject. The processor also outputs a parameter of a pulse sequence for imaging an image of the subject, in a manner suitable for the magnetization relaxation characteristic of the subject, the magnetization relaxation characteristic being based on the acquired age information and relaxation characteristic information. The relaxation characteristic information includes mapping of the age information and the magnetization relaxation characteristic.

### CROSS-REFERENCE TO RELATED APPLICATIONS

This application is based upon and claims the benefit of priority from Japanese Patent Application No. 2019-186184, filed on Oct. 9, 2019; the entire contents of which are incorporated herein by reference.

### FIELD

Embodiments described herein relate generally to a magnetic resonance imaging apparatus and an image processing apparatus.

### BACKGROUND

Conventionally, when an image of a patient is to be imaged using a magnetic resonance imaging apparatus, a technologist or the like sets the imaging parameters based on the conditions of the patient or the region of interest, for example. When an image of the same patient is to be imaged a plurality of number of times with some time therebetween, for example, it is sometimes impossible to use the same imaging parameters as those previously used, because the magnetization relaxation time changes due to the effect of the growth of the patient, for example. In such a case, it sometimes takes a long time to capture an image, because of the time required in setting the imaging parameters.

### BRIEF DESCRIPTION OF THE DRAWINGS

FIG. 1 is a block diagram illustrating one example of a configuration of an MRI apparatus according to a first embodiment;

FIG. 2 is a schematic illustrating one example of an age information input screen according to the first embodiment;

FIG. 3 is a schematic illustrating one example of relaxation characteristic information according to the first embodiment;

FIG. 4 is a graph illustrating one example of a relation between the age information, and a T1 value and a T2 value registered in the relaxation characteristic information according to the first embodiment;

FIG. 5 is a graph illustrating one example of the difference in T1 recovery curves with different T1 values;

FIG. 6 is a graph illustrating one example of the difference in T2 attenuation curves with different T2 values;

FIG. 7 is a schematic illustrating one example of TR for a child according to the embodiment;

FIG. 8 is a table indicating one example of parameters of a pulse sequence, used for each age class, when a longitudinal relaxation emphasized image is to be captured along an axial slice using an SE technique;

FIG. 9 is a table indicating one example of the parameters of a pulse sequence, used for each of the age classes, when a transverse relaxation emphasized image is to be captured along the axial slice using an FSE technique;

FIG. 10 is a schematic illustrating one example of an imaging parameter display screen according to the first embodiment;

FIG. 11 is a flowchart illustrating one example of the sequence of an imaging process according to the first embodiment;

FIG. 12 is a schematic illustrating one example of a configuration of a medical information system according to a second embodiment;

FIG. 13 is a conceptual schematic illustrating one example of MR images captured by an MRI apparatus according to the second embodiment, and calculated images generated by an image processing function; and

FIG. 14 is a flowchart illustrating one example of the sequence of image processing according to the second embodiment.

### DETAILED DESCRIPTION

A magnetic resonance imaging apparatus and an image processing apparatus according to some embodiments will now be explained in detail with reference to some drawings.

### First Embodiment

A magnetic resonance imaging apparatus according to an embodiment includes a processor. The processor is configured to acquire age information representing the length of time elapsed from the birth of the subject. The processor is also configured to output parameters of a pulse sequence for imaging an image of the subject, in a manner suitable for the magnetization relaxation characteristic of the subject, the magnetization relaxation characteristic being based on the acquired age information and relaxation characteristic information. The relaxation characteristic information includes mapping of the age information and the magnetization relaxation characteristic.

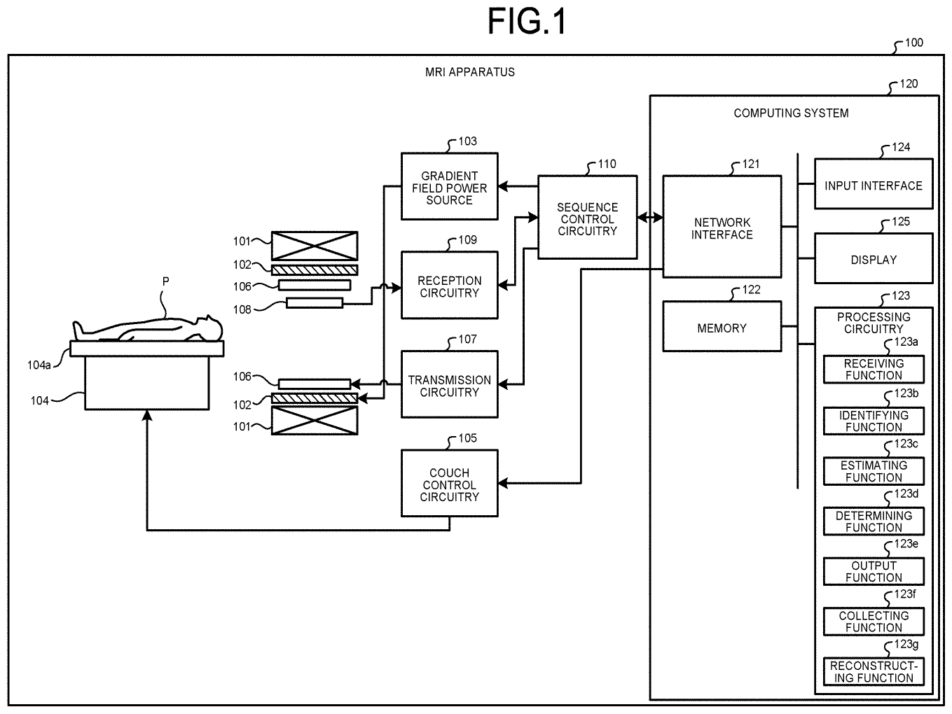

FIG. 1 is a block diagram illustrating one example of a configuration of a magnetic resonance imaging (MRI) apparatus 100 according to the present embodiment.

The MRI apparatus 100 includes a static magnet 101, a gradient coil 102, a gradient field power source 103, a couch 104, couch control circuitry 105, a transmission coil 106, transmission circuitry 107, a reception coil 108, reception circuitry 109, sequence control circuitry 110, a computing system 120, and a gantry 150. The MRI apparatus 100 does not include a subject P. The subject P is a patient an image of which is to be captured with the MRI apparatus 100, for example.

The static magnet 101 is a magnet having a hollowed cylindrical shape (including a shape having an elliptic cross section across a plane perpendicularly intersecting with the axis of the cylinder), and generates uniform static magnetic field in the internal space.

The gradient coil 102 is a coil having a hollowed cylindrical shape (including a shape having an elliptic cross section across a plane perpendicularly intersecting with the axis of the cylinder), and generates a gradient field. The gradient coil 102 is provided as a combination of three coils corresponding to the X, Y, and Z axes, respectively, perpendicularly intersecting with one another, and each of these three coils generates a gradient field the magnetic field intensity of which changes in a direction along the corresponding one of the X, Y, and Z axes, by receiving an independent current supply from the gradient field power source 103.

The gradient field power source 103 supplies a current to the gradient coil 102. For example, the gradient field power source 103 supplies independent currents to the three coils included in the gradient coil 102.

The couch 104 includes a couchtop 104a on which the subject P is laid, and carries the couchtop 104a with the subject P laid thereon into a cavity (imaging opening) of the gradient coil 102, under the control of the couch control circuitry 105. The couch control circuitry 105 is a processor that moves the couchtop 104a in the longitudinal and vertical directions by driving the couch 104, under the control of the computing system 120.

The transmission coil 106 is disposed on the inner side of the gradient coil 102, and applies a high-frequency magnetic field to the subject P by receiving a high-frequency radio frequency (RF) signal from the transmission circuitry 107. Protons in the subject P become excited by receiving the application of the high-frequency magnetic field from the transmission coil 106.

The transmission circuitry 107 supplies an RF pulse corresponding to a Larmor frequency determined based on the type of target atomic nucleus and the magnetic field intensity, to the transmission coil 106, under the control of the sequence control circuitry 110.

The reception coil 108 is disposed on the inner side of the gradient coil 102, and receives a magnetic resonance signal (hereinafter, referred to as an MR signal) emitted from the subject P as a result of being affected by the high-frequency magnetic field. When the MR signal is received, the reception coil 108 outputs the received MR signal to the reception circuitry 109. In the exemplary configuration illustrated in FIG. 1, the reception coil 108 and the transmission coil 106 are provided separately, but this example is merely one example, and the embodiment is not limited to this configuration. For example, it is possible to use a configuration in which the reception coil 108 is also used as the transmission coil 106.

The reception circuitry 109 generates MR data by performing an analog-to-digital conversion to the analog MR signal output from the reception coil 108. The reception circuitry 109 then transmits the generated MR data to the sequence control circuitry 110. It is also possible for the analog-to-digital conversion to be performed by the reception coil 108. The reception circuitry 109 may also perform any other signal processing other than the analog-to-digital conversion.

The sequence control circuitry 110 captures an image of the subject P, by controlling the gradient field power source 103, the transmission circuitry 107, and the reception circuitry 109, based on sequence information received from the computing system 120. The sequence control circuitry 110 also receives MR data from the reception circuitry 109. The sequence control circuitry 110 then transfers the received MR data to the computing system 120.

The sequence control circuitry 110 may be implemented as a processor, or as a combination of software and hardware, for example.

The sequence information is information defining the sequence by which an image is captured. The computing system 120 generates the sequence information based on various parameters that are used in executing of a pulse sequence for imaging an image of the subject P.

A pulse sequence is a sequence of a series of processes for applying an RF pulse or a gradient field to the subject P, and imaging an image of the subject P. For example, in one pulse sequence, a plurality of runs of imaging is performed, correspondingly to the different types of MR images, different imaging methods, or different slices to be imaged. Examples of the types of MR images include a longitudinal relaxation emphasized image (T1 W image), and a transverse relaxation emphasized image (T2 W image).

In the present embodiment, a pulse sequence includes a process for a pre-scan and that for an actual scan. Examples of the pre-scan include a scan for acquiring a low-resolution MR image for determining the region of interest for which an image is to be captured, a scan for acquiring sensitivity information of the reception coil 108, and a scan for acquiring information for correcting the unevenness of the MR image.

The pulse sequence also includes processes such as an application of a pre-pulse, performed precedingly to the imaging of an MR image. The pulse sequence does not need to include a plurality of runs of imaging, and may be the sequence of a process including a single run of imaging.

In the present embodiment, the parameters of a pulse sequence include at least one of repetition time (TR), echo time (TE), and flip angle.

The parameters of a pulse sequence may also include parameters related to the pre-pulse. For example, the parameters of a pulse sequence include inversion time (TI).

The parameters of the pulse sequence also include parameters for imaging T2 maps. A T2 map is an image representing different types of tissues in the subject P in different T2 values. A T2 map is generated by performing a plurality of runs of a process of applying an RF pulse with a different TE and collecting the resultant MR signals, in the pre-scan. The imaging parameters for acquiring a T2 map is information defining the RF pulses and the like used in the pre-scan.

The parameters of a pulse sequence are not limited to the examples described above, and may also include parameters for imaging a T1 map, a slice plane position, a slice thickness, a slice plane inclination, and a field of view (FOV), for example.

When no distinction is to be made among the individual parameters of the pulse sequence used in imaging an image of the subject P, the parameters will be simply referred to as imaging parameters.

The computing system 120 is responsible for controlling the entire MRI apparatus 100, collecting data, and reconstructing images. The computing system 120 includes a network interface 121, a memory 122, processing circuitry 123, an input interface 124, and a display 125.

The network interface 121 transmits sequence information to the sequence control circuitry 110, and receives MR data from the sequence control circuitry 110. The MR data received by the network interface 121 is stored in the memory 122.

The memory 122 stores therein various computer programs. The memory 122 is implemented as a random access memory (RAM), a semiconductor memory device such as a flash memory, a hard disk, or an optical disc, for example. The memory 122 is also used by hardware, as a non-transitory storage medium. The memory 122 is one example of the storage unit.

The memory 122 stores therein relaxation characteristic information. The relaxation characteristic information is information including mapping of age information and a magnetization relaxation characteristic. The relaxation characteristic information will be described later in detail.

The input interface 124 receives various types of instructions and information entered by an operator, such as a physician or a medical radiologist. The input interface 124 is implemented as a trackball, a switch button, a mouse, a keyboard, and the like.

In the present embodiment, the input interface 124 is not limited to those provided with a physical operational component, such as a mouse and a keyboard. Another example of the input interface 124 includes electric signal processing circuitry that receives an electric signal corresponding to an input operation from an external input device provided separately from the MRI apparatus 100, and that outputs the electric signal to the processing circuitry 123.

The input interface 124 is connected to the processing circuitry 123, converts various input operations received from the operator into electric signals, and outputs the resultant electric signals to the processing circuitry 123.

The display 125 displays various graphical user interfaces (GUIs), magnetic resonance (MR) images, or various types of images generated by the processing circuitry 123, for example, under the control of the processing circuitry 123. The display 125 is one example of the display unit.

The processing circuitry 123 controls the entire MRI apparatus 100. More specifically, the processing circuitry 123 includes a receiving function 123a, an identifying function 123b, an estimating function 123c, a determining function 123d, an output function 123e, a collecting function 123f, and a reconstructing function 123g. The receiving function 123a is one example of a receiving unit. The identifying function 123b is one example of identifying unit. The estimating function 123c is one example of an estimating unit. The determining function 123d is one example of a determining unit. The output function 123e is one example of an output unit. The collecting function 123f is one example of a collecting unit. The reconstructing function 123g is one example of a reconstructing unit.

These processing functions including the receiving function 123a, the identifying function 123b, the estimating function 123c, the determining function 123d, the output function 123e, the collecting function 123f, and the reconstructing function 123g, which are the elements of the processing circuitry 123, are stored in the memory 122 as computer-executable programs, for example. The processing circuitry 123 implements the functions corresponding to the respective computer programs by reading the computer programs from the memory 122, and executing the computer programs. In other words, the processing circuitry 123 having read the computer programs has functions illustrated inside of the processing circuitry 123 in FIG. 1. Explained in FIG. 1 is an example in which the processing functions including the receiving function 123a, the identifying function 123b, the estimating function 123c, the determining function 123d, the output function 123e, the collecting function 123f, and the reconstructing function 123g are implemented by one processing circuitry 123, but as another implementation of the processing circuitry 123, it is possible to combine a plurality of independent processors, and to cause each of the processors to execute corresponding one of the computer programs.

The term “processor” used in the explanation above means circuitry such as a central processing unit (CPU), a graphics processing unit (GPU), an application specific integrated circuit (ASIC), or a programmable logic device (such as a simple programmable logic device (SPLD), a complex programmable logic device (CPLD), and a field programmable gate array (FPGA)). It is also possible to incorporate the computer programs directly into the processor circuitry, instead of storing the computer programs in the memory 122. In such a case, the processor implements the functions by reading computer programs incorporated therein, and executing the computer programs.

The receiving function 123a receives various operations made by the operator, via the input interface 124. For example, the receiving function 123a acquires age information by receiving an input of age information that represents the length of time elapsed from the birth of the subject P, the age information being entered by the operator. Hereinafter, in the present embodiment, “acquiring” means both of receiving, as an input from a user, and receiving over a network or the like.

The age information is information representing the time elapsed from the birth of the subject P to the time of imaging. In the present embodiment, the age information is information representing the monthly age of the subject P. In the present embodiment, the “monthly age” means information representing the time elapsed from the birth of the subject P to now in units of months. More specifically, age information is information representing the time elapsed from the birth of the subject P to the time of imaging for a subject P who is in his/her childhood.

In the present embodiment, a “child” is defined as a person who is younger than 15 years old, for example. Furthermore, in the present embodiment, any person equal to or older than 15 years old will be referred to as an “adult”. Furthermore, in the present embodiment, to classify children into smaller age classes, a child who is younger than 4 weeks old will be referred to as a “newborn”, a child who is younger than 1 year old will be referred to as an “infant”, and a child who is younger than 6 years old will be referred to as a “preschool child”, for example. When the term “child” alone is used, the term is inclusive of the “newborn”, the “infant”, and the “preschool child”. However, these definitions are merely one example, and the definitions of the ages of children classified into each of the age classes may be changed depending on the operation.

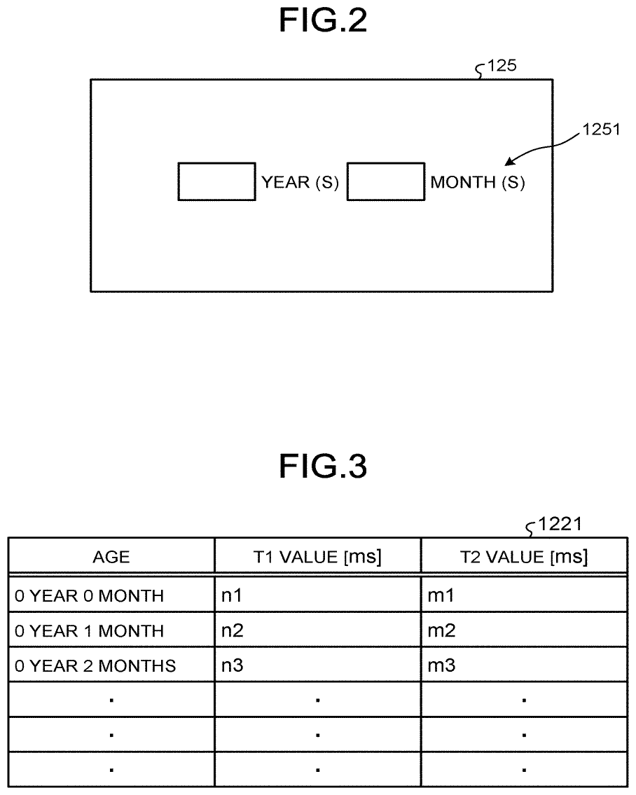

FIG. 2 is a schematic illustrating one example of an input screen 1251 via which the age information is entered in the embodiment. In this example, the output function 123e, which will be described later, displays the input screen 1251 on the display 125, for example. As illustrated in FIG. 2, the input screen 1251 is a screen where the age of the subject P can be entered in units of one month. The receiving function 123a receives the age information entered in the input screen 1251, via the input interface 124. The receiving function 123a sends the received age information to the identifying function 123b.

The receiving function 123a receives information related to a method for imaging an image of subject P, entered by the operator, via the input interface 124. Examples of the imaging method include a spin echo (SE) technique and a fast spin echo (FSE) technique. The receiving function 123a sends the received information related to the imaging method to the determining function 123d.

The receiving function 123a receives the values of the imaging parameters input via an imaging parameter display screen that is displayed by the output function 123e, which will be described later. The receiving function 123a sends the received values of the imaging parameters to the output function 123e and the collecting function 123f. The imaging parameter display screen will be described later in detail.

The receiving function 123a receives an imaging starting operation entered by the operator. When the imaging starting operation is received, the receiving function 123a sends information indicating that the operation is received to the collecting function 123f. The operations receivable by the receiving function 123a is not limited thereto, and, for example, the receiving function 123a receives operations for setting the FOV or the slice position, entered by the operator, and an operation for starting the actual scan after the pre-scan is executed, for example.

Referring back to FIG. 1, the identifying function 123b identifies the magnetization relaxation characteristic of the subject P based on the age information received by the receiving function 123a and the relaxation characteristic information stored in the memory 122.

The magnetization relaxation characteristic in the present embodiment is a T1 value or a T2 value, for example. A T1 value represents time required for the magnetization vector in the vertical direction to recover to approximately 63% of the original value, from the time when the protons of the subject P have become excited, for example. A T2 value represents time required for the magnetization vector in the lateral direction to attenuate to approximately 37% from its maximum value, from the time when the protons of the subject P have become excited, for example. The T1 value and the T2 value are represented in milliseconds (ms), for example.

In the present embodiment, the “recovery” of the magnetization vector in the vertical direction and the “attenuation” of the magnetization vector in the lateral direction will be collectively referred to as “relaxation”. To refer to the T1 value and the T2 values collectively, the term “relaxation time” will be used.

FIG. 3 is a schematic illustrating one example of relaxation characteristic information 1221 according to the embodiment. As illustrated in FIG. 3, in the relaxation characteristic information 1221, an age is mapped to a T1 value and a T2 value.

In the example of the present embodiment, the T1 values and the T2 values registered in the relaxation characteristic information 1221 are those of a type of brain tissue. Specifically, the T1 values and the T2 values registered in the relaxation characteristic information 1221 are those of the white matter or the gray matter, for example. Furthermore, a plurality of pieces of the relaxation characteristic information 1221 may be stored in the memory 122 correspondingly to the types of tissues. In such relaxation characteristic information 1221, an age is registered in a manner mapped to a T1 value and a T2 value of the white matter, and to those of the gray matter, for example.

Furthermore, the T1 value and the T2 value in the relaxation characteristic information 1221 may also be the T1 value and the T2 value that are typical of the age. The T1 value and T2 value that are typical of the age may be values calculated by applying statistical process to detection results collected from a plurality of subjects P, for example.

More specifically, it is assumed that mapped in the relaxation characteristic information 1221 according to the present embodiment are the age information and the T1 value and the T2 value of the subjects who are in the childhood. However, the entire age information included in the childhood does not need to be registered in the relaxation characteristic information 1221. For example, the age information corresponding to at least from the birth to the preschool period may be registered in the relaxation characteristic information 1221, in a manner mapped to their T1 value and T2 value.

This is because the T1 value and the T2 value of a child change greatly depending on his/her age, as the child develops (comes into maturity). In particular, the T1 value and the T2 value of a child's brain change greatly depending on his/her age, due to the effect of the formation of myelin sheaths (white matter), and the development of synapses (gray matter). By contrast, the T1 value and the T2 value of the brain of a developed adult goes through a small change depending on his/her age. Therefore, it is possible not to register the T1 value and the T2 value in the relaxation characteristic information 1221 for adults whose age has passed the childhood.

In FIG. 3, the relaxation characteristic information 1221 is illustrated in a database format, but the format of the relaxation characteristic information 1221 is not limited thereto.

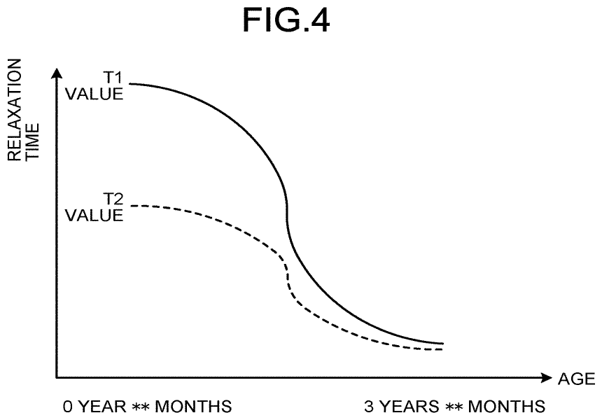

FIG. 4 is a graph illustrating one example of a relation between the age information, and the T1 value and the T2 value registered in the relaxation characteristic information 1221 according to the embodiment. The vertical axis in FIG. 4 represents the relaxation time, and the horizontal axis represents age. As one example, the T1 value and the T2 value registered in the relaxation characteristic information 1221 explained with reference to FIG. 3 attenuate as illustrated in FIG. 4, as the age increases.

The T1 value and the T2 value exhibit different values depending on the tissues of a human body, but the T1 value and the T2 value registered in the relaxation characteristic information 1221 are those of a specific type of tissues.

The identifying function 123b searches the relaxation characteristic information 1221 for an age finding a match with the age specified in the age information received by the receiving function 123a, and identifies the T1 value and the T2 value that are mapped to the matching age, as the T1 value and the T2 value of the subject P. The identifying function 123b sends the identified T1 value and T2 value to the estimating function 123c.

Referring back to FIG. 1, the estimating function 123c estimates a magnetization relaxation curve for the subject P, based on the magnetization relaxation characteristic identified by the identifying function 123b. The magnetization relaxation curve is a T1 recovery curve or a T2 attenuation curve.

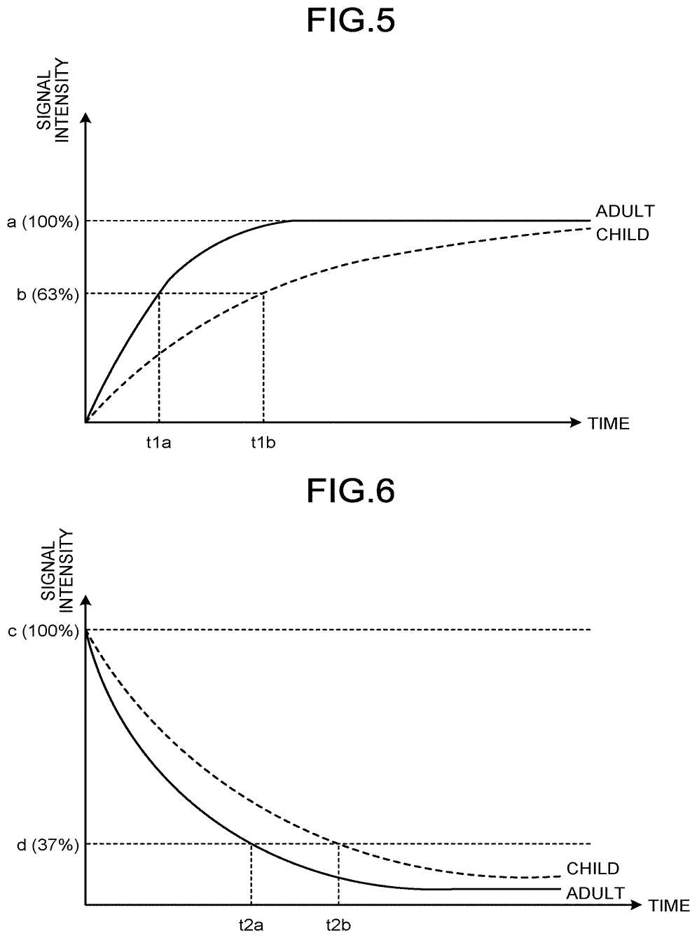

FIG. 5 is a graph illustrating one example of the difference in the T1 recovery curves with different T1 values. The vertical axis in FIG. 5 represents a signal intensity of the MR signal, and the horizontal axis represents time. In FIG. 5, “a” indicates the signal intensity at the time when the horizontal relaxation recovers to 100%. “b” indicates the signal intensity at the time when the magnetization vector in the vertical direction recovers to approximately 63% of the original value. A T1 value is representative of the relaxation time required for the signal intensity to reach “b”.

The estimating function 123c estimates a T1 recovery curve based on the T1 value identified by the identifying function 123b.

In FIG. 5, the T1 value of a certain type of tissues of a typical adult is plotted as “t1a”, and the T1 value of a certain type of tissues of a person whose age is within the childhood is plotted as “t1b”. It is assumed herein that the types of the tissues of the child and of the adult illustrated in FIG. 5 are the same. Because the T1 value of the child is greater than that of an adult, as explained with reference to FIG. 4, the T1 recovery curve of the child has a gentler gradient than that of the adult.

The estimating function 123c also estimates a T2 attenuation curve based on the T2 value identified by the identifying function 123b.

FIG. 6 is a graph illustrating one example of the difference in the T2 attenuation curves with different T2 values. The vertical axis in FIG. 6 represents a signal intensity of the MR signal, and the horizontal axis represents time. In FIG. 6, “c” indicates the highest signal intensity reached after the excitation of the magnetization vector in the lateral direction. “d” indicates the signal intensity of the magnetization vector in the lateral direction, having attenuated to approximately 37%, with respect to 100% that is the highest intensity after the excitation of the magnetization vector in the lateral direction.

In FIG. 6, the typical T2 value of a certain type of tissues of a subject P who is an adult is plotted as “t2a”, and the typical T2 of a certain type of tissues of a subject P whose age is within the childhood is plotted as “t2b”. It is assumed herein that the types of tissues of the child and of the adult illustrated in FIG. 6 are the same. Because the T2 value of a child is greater than that of an adult, the T2 attenuation curve of the child has a gentler gradient than that of the adult.

The estimating function 123c estimates the T1 recovery curve and the T2 attenuation curve for each type of tissues for which images are to be captured, for example. When the region of interest is a brain, for example, the estimating function 123c estimates the T1 recovery curve and the T2 attenuation curve for the white matter, as well as for the gray matter. Alternatively, the estimating function 123c may be configured to estimate the T1 recovery curve and the T2 attenuation curve of a typical type of tissues. The estimating function 123c then sends the estimated T1 recovery curves and T2 attenuation curves to the determining function 123d.

Referring back to FIG. 1, the determining function 123d determines the parameters of a pulse sequence to be used in imaging of the subject P, based on the relaxation curves estimated by the estimating function 123c.

As mentioned earlier, the T1 value and the T2 value of a child are greater than those of an adult. The contrast in the magnetic resonance (MR) images are affected by both of the T1 value and the T2 value. Therefore, if the same parameters are used for a child, as those used for capturing images from an adult, for example, there are cases that the contrast becomes insufficient.

Even in the childhood, the T1 value and the T2 value go through a dramatic change within a period of growing from a newborn to a preschool child, and there are times when the T1 value and the T2 value goes through a significant change within a few months, for example. Therefore, the imaging parameters enabling capturing of MR images with appropriate contrast change depending on the age, even if the image to be captured is of the same subject P.

For example, the determining function 123d determines the parameters, such as TR and TE, achieving sufficient contrast between a plurality of types of tissues in the MR image, based on the imaging method to be used to capturing the image of the subject P, and on the relaxation curves corresponding to the types of tissues, estimated by the estimating function 123c. The determining function 123d acquires the imaging method to be used to capture the image of the subject P, from the receiving function 123a.

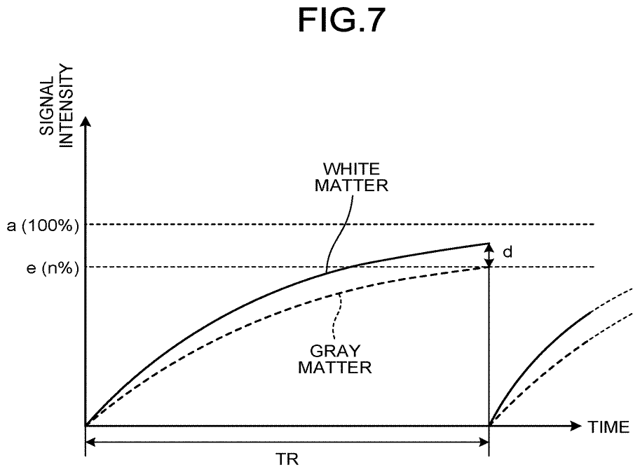

FIG. 7 is a schematic illustrating one example of TR for a child according to the embodiment. Illustrated in FIG. 7, as one example, are the T1 recovery curves for the white matter and the gray matter of a subject P who is a child. As illustrated in FIG. 7, because, among the tissues of a brain, the gray matter contains a larger amount of water than white matter does, the TR of the gray matter is longer than that of the white matter, and therefore, the T1 recovery curve of the gray matter has a gentler gradient than that of the white matter.

The determining function 123d determines the length of time from when the RF pulse is applied to when following two conditions are satisfied as TR, for example: a difference d between the T1 recovery curve of the gray matter and that of the white matter has reached a specified amount; and the signal intensity has recovered to a percentage equal to or higher than a predetermined percentage. In FIG. 7, the predetermined percentage is denoted as n %. The specified amount of the difference d between the recovery curve T1 of the gray matter and that of the white matter is set to an amount allowing sufficient contrast to be achieved among different types of tissues in the MR image.

Because the amount of water in the brain tissues of a child is different from that in the brain tissues of an adult, the recovery curves T1 of the white matter and the gray matter of a subject P who is a child are different from those of an adult. Therefore, when an MR image to be captured is an image of the head of a child, TR for achieving an image with appropriate contrast is different from that when an MR image to be captured is an image of the head of an adult.

In FIG. 7, although the T1 recovery curves are illustrated as an example, the determining function 123d also considers the T2 attenuation curves, as well as the T1 recovery curves, into consideration, in determining TR and TE for achieving sufficient contrast between different types of tissues, suitable for the imaging method. Examples of the imaging method include types of MR images, such as a longitudinal relaxation emphasized image (T1 W image) and a transverse relaxation emphasized image (T2 W image), and an excitation method such as the spin echo (SE) technique and the fast spin echo (FSE) technique.

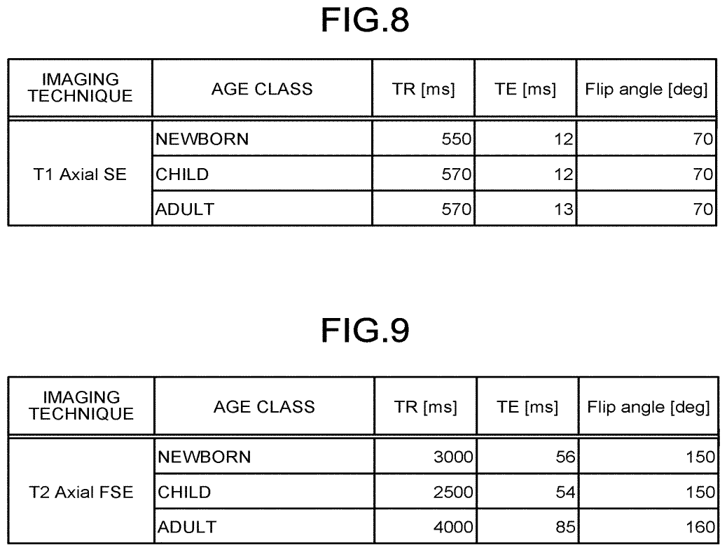

Explained now specifically with reference to FIGS. 8 and 9 is how different imaging parameters are used for different ages.

FIG. 8 is a table indicating one example of the parameters of a pulse sequence, used for each of the age classes, when a longitudinal relaxation emphasized image (T1 W image) is to be captured along an axial slice (a slice along the body axis) using the SE technique. In FIG. 8, TR, TE, and flip angle are indicated as examples of the imaging parameters. In the longitudinal relaxation emphasized image, TR is particularly affected by the T1 value. In the imaging method indicated in FIG. 8, for example, when an image to be captured is an image of a “newborn”, TR for achieving appropriate contrast is set to “550”, and is smaller than “570” that is TR for a “child” and an “adult”. This means that, with the imaging method indicated in FIG. 8, to achieve the contrast equivalent to that achieved when an image of an adult is to be captured, TR shorter by 20 ms is used for a newborn.

FIG. 9 is a table indicating one example of the parameters of a pulse sequence, used for each of the age classes, when a transverse relaxation emphasized image (T2 W image) is to be captured along the axial slice using the FSE technique. In a transverse relaxation emphasized image, TE is particularly affected by the T2 value. In the imaging method indicated in FIG. 9, when an image to be captured is an image of a “newborn”, for example, TE for achieving appropriate contrast is set to “56”, and is smaller than “85” that is TE for an “adult”. Furthermore, TE for achieving appropriate contrast when an image of a “child” is to be captured is set to “54”, and is smaller than those of “adult” and “newborn”. This means that, with the imaging method indicated in FIG. 9, to achieve the contrast equivalent to that achieved in an image of an adult, TE shorter by 29 ms is used for a newborn, and TE shorter by 31 ms is used for a child.

The numbers indicated in FIGS. 8 and 9 are just some examples, and values of the parameters are not limited to these numbers indicated therein. In FIGS. 8 and 9, the age is classified into three classes of “newborn”, “child”, and “adult”, but this is just one example, and it also does not mean that the same parameters are applied to all of the subjects P whose ages fall within the corresponding age class. Furthermore, in FIGS. 8 and 9, “newborns” are excluded from the age class “child”.

The determining function 123d then sends the determined parameters to the output function 123e.

Referring back to FIG. 1, the output function 123e outputs the imaging parameters that are based on the age information received by the receiving function 123a, and on the relaxation characteristic information 1221. More specifically, the output function 123e displays the imaging parameters determined by the determining function 123d on the display 125.

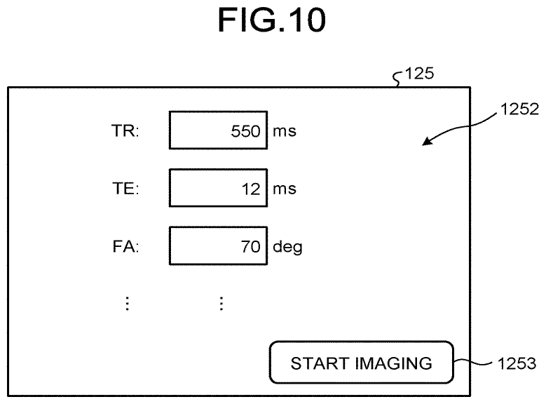

FIG. 10 is a schematic illustrating one example of an imaging parameter display screen 1252 according to the embodiment. As illustrated in FIG. 10, the output function 123e displays the imaging parameters such as TR, TE, and FA in the imaging parameter display screen 1252, on the display 125.

The values of the imaging parameters displayed in the imaging parameter display screen 1252 are configured changeable. The operator can therefore change the values of imaging parameters in the imaging parameter display screen 1252, using the input interface 124, for example. The values of the imaging parameters entered by the operator are received by the receiving function 123a.

The output function 123e also displays an imaging start button 1253 capable of receiving an imaging starting operation made by the operator, in the imaging parameter display screen 1252.

The types and the values of the imaging parameters indicated in FIG. 10 are merely exemplary, and the embodiment is not limited thereto.

The output function 123e also displays a reference MR image and a diagnostic MR image generated by the reconstructing function 123g, on the display 125.

The reference MR image is an MR image generated by the reconstructing function 123g, based on the result of the pre-scan performed by the collecting function 123f, which will be described later. The reference MR image has a lower resolution than the diagnostic MR image captured in the actual scan. The reference MR image may also have a function of a positioning image for receiving inputs such as an FOV or a slice position from the operator.

Referring back to FIG. 1, the collecting function 123f collects MR data resultant of executing various pulse sequences, and of converting the MR signal acquired through the execution of the pulse sequences, from the sequence control circuitry 110, via the network interface 121.

More specifically, the collecting function 123f generates sequence information based on the imaging parameters determined by the determining function 123d, or on the imaging parameters received by the receiving function 123a. For example, when the values of the imaging parameters have been changed by the operator, the collecting function 123f generates sequence information using the changed values of the imaging parameters. When the values of the imaging parameters have not been changed by the operator, the collecting function 123f generates sequence information based on the imaging parameters determined by the determining function 123d.

The collecting function 123f then sends the generated sequence information to the sequence control circuitry 110 via the network interface 121. By causing the sequence control circuitry 110 to execute a process based on the sequence information, for example, a pre-scan or an actual scan defined in the sequence information is executed.

In the present embodiment, the pre-scan and the actual scan are not executed continuously. For example, when the operator makes an operation for starting the actual scan after the reference MR image is displayed as a result of the pre-scan, the collecting function 123f starts the actual scan. When the operator has changed the imaging parameters based on the result of the pre-scan, the collecting function 123f changes the sequence information based on the changed imaging parameters, and sends the changed sequence information to the sequence control circuitry 110.

The collecting function 123f also plots the MR data collected as a result of executing the pre-scan or the actual scan, based on the amount of phase-encoding or the amount of frequency encoding achieved by the gradient field. The MR data plotted in the k-space is referred to as k-space data. The k-space data is stored in the memory 122.

The reconstructing function 123g generates an MR image by applying a reconstructing process such as Fourier transform to the k-space data stored in the memory 122. The reconstructing function 123g then stores the generated MR image in the memory 122.

More specifically, the reconstructing function 123g generates a reference MR image based on the k-space data generated from the MR data acquired in the pre-scan. The reconstructing function 123g then sends the generated reference MR image to the output function 123e.

The sequence of an imaging process performed by the MRI apparatus 100, having the configuration described above, will now be explained. The imaging process includes an imaging parameter generating process.

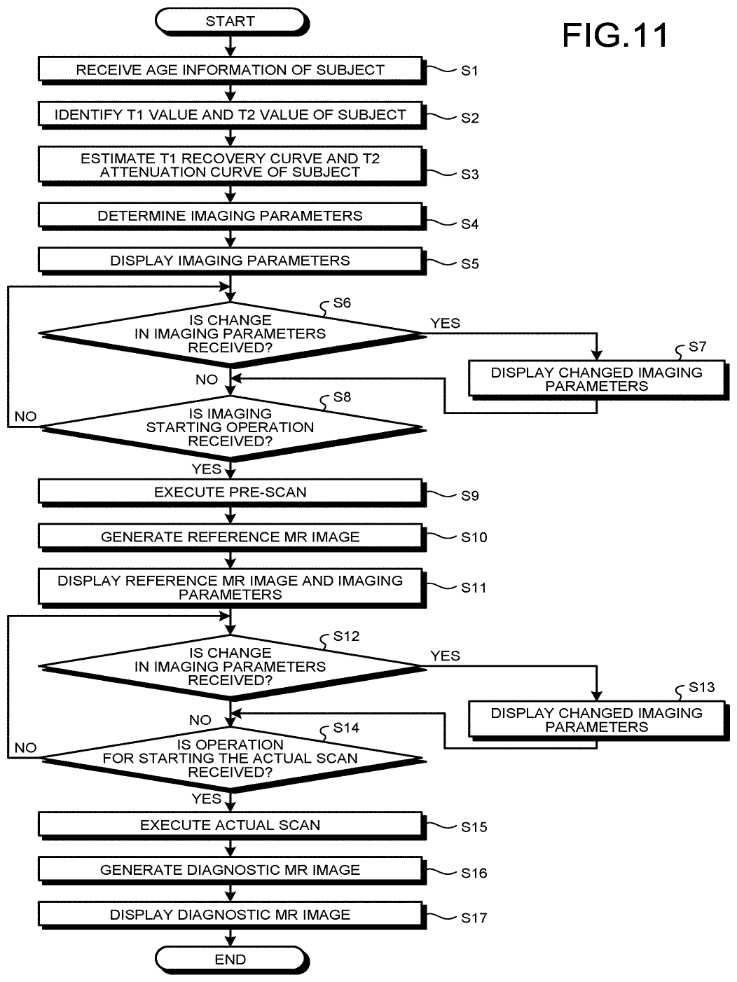

FIG. 11 is a flowchart illustrating one example of the sequence of an imaging process according to the embodiment.

To begin with, the receiving function 123a receives the age information of the subject P input to the input screen 1251 by an operator (S1). For example, when the subject P is a child, the operator enters the monthly age of the subject P as the age information. The receiving function 123a then sends the received age information to the identifying function 123b.

The identifying function 123b then identifies the T1 value and the T2 value of the subject P based on the age information received by the receiving function 123a, and the relaxation characteristic information 1221 stored in the memory 122 (S2). The identifying function 123b then sends the identified T1 value and T2 value to the estimating function 123c.

The estimating function 123c estimates the T1 recovery curve and the T2 attenuation curve of the subject P, from the T1 value and the T2 value identified by the identifying function 123b (S3). The estimating function 123c then sends the estimated T1 recovery curve and T2 attenuation curve to the determining function 123d.

The determining function 123d determines the imaging parameters based on the T1 recovery curve and the T2 attenuation curve estimated by the estimating function 123c (S4). The determining function 123d then sends the determined imaging parameters to the output function 123e and the collecting function 123f.

The output function 123e then displays the imaging parameters determined by the determining function 123d, in the imaging parameter display screen 1252 on the display 125 (S5).

The receiving function 123a then determines whether a change in the imaging parameters in the imaging parameter display screen 1252 has been received from the operator (S6). If it is determined that a change in the imaging parameters has been received (Yes at S6), the receiving function 123a sends the changed values of the imaging parameters to the output function 123e. When this is the case, the output function 123e displays the changed imaging parameters on the imaging parameter display screen 1252 (S7). After the process at S7, the system control goes to the process at S8. Even if it is determined that any change in the imaging parameters has not been received (No at S6), the receiving function 123a goes to the process at S8.

The receiving function 123a then determines whether an imaging starting operation has been received from the operator (S8). For example, the receiving function 123a determines that an imaging starting operation has been received from the operator, if the imaging start button 1253 in the imaging parameter display screen 1252 has been pressed down by the operator (Yes at S8). If it is determined that the imaging start button 1253 has not been pressed down (No at S8), the receiving function 123a repeats the process at S6 to S8.

If it is determined that the imaging starting operation has been received from the operator, the receiving function 123a sends a notification of the reception of the operation, to the collecting function 123f. When this is the case, the collecting function 123f generates the sequence information based on the latest values of the imaging parameters. The collecting function 123f then sends the generated sequence information to the sequence control circuitry 110 via the network interface 121, and starts executing the pre-scan (S9). The collecting function 123f then generates k-space data from the MR data that is collected as a result of the execution of the pre-scan.

The reconstructing function 123g then generates a reference MR image by applying a reconstructing process such as Fourier transform to the generated k-space data (S10). The reconstructing function 123g sends the generated reference MR image to the output function 123e.

The output function 123e then displays the reference MR image and the imaging parameters to be used in the actual scan on the display 125 (S11).

The receiving function 123a then determines whether a change in the imaging parameters has been received from the operator (S12). If it is determined that a change in the imaging parameters has been received (Yes at S12), the receiving function 123a sends the changed values of the imaging parameters to the output function 123e. When this is the case, the output function 123e displays the changed imaging parameters on the display 125 (S13). After the process at S13, the system control goes to the process at S14. Even if it is determined that any change in the imaging parameters has not been received (No at S12), the receiving function 123a goes to the process at S14.

The receiving function 123a then determines whether the operation for starting the actual scan has been received from the operator (S14). If it is determined that the operation for starting the actual scan has not been received (No at S14), the receiving function 123a repeats the process at S12 to S14.

If it is determined that the operation for starting the actual scan has been received from the operator (Yes at S14), the receiving function 123a sends a notification of the reception of the operation, to the collecting function 123f. If any of the imaging parameters has been changed after the pre-scan, the collecting function 123f changes the sequence information based on the changed imaging parameters. If none of the imaging parameters has been changed after the pre-scan, the collecting function 123f does not need to change the sequence information.

The collecting function 123f may generate the sequence information for the pre-scan and the sequence information for the actual scan at different timing. In other words, the collecting function 123f may generate only the sequence information for the pre-scan before executing the pre-scan, and then generates sequence information for the actual scan before executing the actual scan, by applying corrections to the imaging parameters based on the result of the pre-scan.

The collecting function 123f then sends the generated sequence information to the sequence control circuitry 110 via the network interface 121, and executes the actual scan (S15). The collecting function 123f then generates k-space data from the MR data collected as a result of the execution of the actual scan.

The reconstructing function 123g then generates a diagnostic MR image by applying a reconstructing process such as Fourier transform to the generated k-space data (S16). The reconstructing function 123g then sends the generated diagnostic MR image to the output function 123e.

The output function 123e then displays the generated diagnostic MR image on the display 125 (S17). The process illustrated in the flowchart is then ended.

As mentioned earlier, the T1 value and the T2 value of the brain of a child change greatly depending on the age, due to the effect of the formation of myelin sheaths (white matter), and the development of synapses (gray matter). Therefore, when the subject P is a child, it is sometimes difficult to capture an MR image having sufficient contrast using the imaging parameters for an adult. Furthermore, when an image of the same subject P is to be captured a plurality of number of times, with some time therebetween, it is sometimes difficult to capture an MR image having sufficient contrast using the same imaging parameters previously used, due to the effect of the growth of the subject P, for example. Therefore, conventionally, there has been times that imaging time becomes extended, because a long time is required for the operator to set the imaging parameters, or the operator is required to re-do the imaging.

By contrast, because the MRI apparatus 100 according to the embodiment outputs the imaging parameters that are based on the age information of the subject P received from the operator, and on the relaxation characteristic information 1221, the operator can easily get a grasp of the imaging parameters suitable for the age of the subject P. Therefore, with the MRI apparatus 100 according to the embodiment, it is possible to reduce the time required in setting the parameters used in imaging an MR image.

More specifically, the MRI apparatus 100 according to the embodiment estimates a magnetization relaxation curve for the subject P from the magnetization relaxation characteristic of the subject P identified based on the age information of the subject P received from the operator, and on the relaxation characteristic information 1221. Therefore, it is possible to determine the imaging parameters corresponding to the age information of the subject P.

Furthermore, the age information according to the embodiment is information indicating the monthly age of the subject P. For example, when the subject P is in the process of growing up, the T1 value and the T2 value can go through a significant change within a few months. When this is the case, with the MRI apparatus 100 according to the embodiment, by setting the imaging parameters corresponding to the age of the subject P in units of one month, it is possible to set the imaging parameters for achieving an MR image having appropriate contrast.

Furthermore, the age information according to the embodiment represents the age of the subject who is in the childhood from his/her birth to the time of imaging. Furthermore, the magnetization relaxation characteristic according to the embodiment is a T1 or T2 value, and the relaxation characteristic information 1221 includes mapping of the age information and a T1 or T2 value of a subject P who is a child. When the subject P is an adult, the T1 value and the T2 value are less affected by his/her age. By contrast, when the subject P is a child, the T1 value and the T2 value may change greatly depending on the time elapsed from his/her birth to the time of imaging. Therefore, by presenting the imaging parameters corresponding to the age information, at least for a subject who is in the childhood, the MRI apparatus 100 according to the embodiment can keep up with a change in the magnetization relaxation characteristic caused by the growth of the subject P.

Furthermore, the imaging parameters according to the embodiment includes at least one of TR, TE, and flip angle. By setting values based on the age information of the subject P to at least one of TR, TE, and flip angle that are major imaging parameters used in imaging images of the subject P, the MRI apparatus 100 according to the embodiment can execute an imaging process suitable for the magnetization relaxation characteristic of the subject P, the magnetization relaxation characteristic being affected by a change in the age.

Furthermore, the imaging parameters according to the embodiment may be TI. Furthermore, the imaging parameters according to the embodiment may also be the imaging parameters for a T2 map. The MRI apparatus 100 according to the embodiment can perform a process suitable for the magnetization relaxation characteristic of the subject P, the magnetization relaxation characteristic being affected by a change in the age, not only for TR, TE, and flip angle used in the process of imaging an MR image, but also for various processes performed as a pre-process preceding the imaging.

In the present embodiment, the operator inputs the age information when the subject P is a child, but the operator may also enter the age information when the subject P is an adult.

Furthermore, in the present embodiment, the age information is explained to be monthly age, but the age information is not limited thereto. For example, the age information may be classification such as newborn, infant, preschool child, child, and adult. Alternatively, the age information may also be represented as a weekly age or a yearly age.

Furthermore, the receiving function 123a may also acquire the age of the subject P from a hospital information system (HIS) or a radiology information system (RIS) external to the MRI apparatus 100.

Furthermore, the method for estimating a T1 recovery curve is not limited to the example described above, and, for example, a T1 recovery curve may be stored in the memory 122 in a manner mapped to a T1 value. When this is the case, the estimating function 123c searches the memory 122 for the T1 value identified by the identifying function 123b, and acquires the T1 recovery curve mapped to the T1 value, as an estimation of the T1 recovery curve of the subject P.

Furthermore, explained in the present embodiment is an example in which the identifying function 123b identifies the T1 value and the T2 value of the subject P, but it is also possible to identify one of the T1 value and the T2 value. Furthermore, the estimating function 123c may also be configured to estimate one of the T1 recovery curve and the T2 attenuation curve.

Furthermore, the estimating function 123c may be configured to adjust the T1 recovery curve and the T2 attenuation curve based not only on the age information, but also on the height, the weight, the sex, the speed of growth, or the like of the subject P. For example, when the height or the weight of the subject P is greater than the average of those of the same monthly age (e.g., n months) as the subject P, the estimating function 123c adjusts to bring the T1 recovery curve and the T2 attenuation curve closer to those of a monthly age older than that of the subject P (e.g., n+1 months). Alternatively, the determining function 123d may adjust the values of the imaging parameters by taking the height, the weight, the sex, the speed of growth, or the like of the subject P into consideration.

Furthermore, the MRI apparatus 100 may be provided with a function for automatically correcting the imaging parameters for the actual scan based on the result of the pre-scan. For example, the determining function 123d may recognize the contrast of the reference MR image generated as a result of a pre-scan, and correct the imaging parameters for the actual scan based on the contrast. For example, when the contrast is lower than a reference value, the determining function 123d may correct the imaging parameters by changing TR, TE, or the like, in such a manner that the contrast is increased. Furthermore, when a plurality of diagnostic MR images are to be captured in the actual scan, the determining function 123d may correct the imaging parameters, based on the contrast of one captured diagnostic MR image, for the other diagnostic MR images to be captured subsequently to the one diagnostic MR image.

Furthermore, explained in the present embodiment is an example in which the determining function 123d determines the imaging parameters based on the T1 recovery curve and the T2 attenuation curve estimated by the estimating function 123c, but the technique for determining the imaging parameters is not limited thereto. For example, it is possible to use a configuration in which the age information is stored in the memory 122 in a manner mapped to various imaging parameters, in advance. When such a configuration is used, the determining function 123d may search the memory 122 for the imaging parameters mapped to the age information received by the receiving function 123a, and determine the found imaging parameters as the imaging parameters of the subject P.

Furthermore, explained in the present embodiment is an example in which the output function 123e displays the imaging parameters determined by the determining function 123d on the display 125, but the mode of the output is not limited thereto. For example, the output function 123e may output the imaging parameters determined by the determining function 123d to the sequence control circuitry 110. When such a configuration is used, the sequence control circuitry 110 performs the sequence for imaging an image of the subject P based on the imaging parameters determined by the determining function 123d. When this is the case, the MRI apparatus 100 may capture an image of the subject P based on the imaging parameters determined by the determining function 123d, without receiving a change of the imaging parameters from the operator.

Furthermore, explained in the embodiment is an example in which the region of interest is a brain, but the configuration according to the embodiment may be applied to imaging of another region of interest. Furthermore, the memory 122 may store therein a plurality of pieces of the relaxation characteristic information 1221, correspondingly to the respective regions of interest or respective types of tissues.

### Second Embodiment

Explained in the first embodiment is how the imaging parameters are set based on the age of the subject P, when an image of the subject P is to be captured using an ordinary MRI apparatus 100. In this second embodiment, however, it is assumed that the MRI apparatus is a synthetic MRI apparatus, and the settings of parameters to be used in image processing are acquired based on the age of the subject P, for the purpose of generating a plurality of different MR images from the result of imaging images using the synthetic MRI.

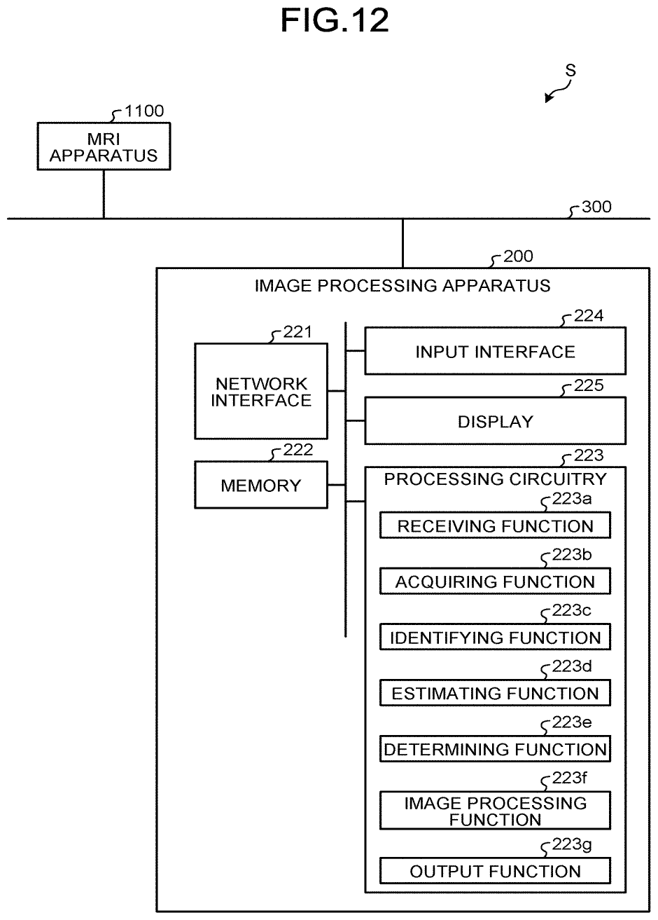

FIG. 12 is a schematic illustrating one example of a configuration of a medical information system S according to the embodiment. As illustrated in FIG. 12, the medical information system S includes an MRI apparatus 1100 and an image processing apparatus 200. The MRI apparatus 1100 and the image processing apparatus 200 are connected to each other over a network 300, such as a local area network (LAN) in a hospital, for example.

The MRI apparatus 1100 is a synthetic MRI apparatus. Furthermore, the MRI apparatus 1100 includes, in the same manner as in the first embodiment, the static magnet 101, the gradient coil 102, the gradient field power source 103, the couch 104, the couch control circuitry 105, the transmission coil 106, the transmission circuitry 107, the reception coil 108, the reception circuitry 109, the sequence control circuitry 110, the computing system 120, and the gantry 150.

The image processing apparatus 200 is a personal computer (PC) or a server, for example. The image processing apparatus 200 includes a network interface 221, a memory 222, a processing circuitry 223, an input interface 224, and a display 225.

The processing circuitry 223 of the image processing apparatus 200 includes a receiving function 223a, an acquiring function 223b, an identifying function 223c, an estimating function 223d, a determining function 223e, an image processing function 223f, and an output function 223g. The receiving function 223a is one example of the receiving unit. The acquiring function 223b is one example of an acquiring unit. The identifying function 223c is one example of the identifying unit. The estimating function 223d is one example of the estimating unit. The determining function 223e is one example of the determining unit. The image processing function 223f is one example of an image processing unit. The output function 223g is one example of the output unit.

These processing functions including the receiving function 223a, the acquiring function 223b, the identifying function 223c, the estimating function 223d, the determining function 223e, the image processing function 223f, and the output function 223g, which are elements of the processing circuitry 223, are stored in the memory 222 as computer-executable programs, for example. The processing circuitry 223 implements the functions corresponding to the respective computer programs by reading the computer programs from the memory 222, and executing the computer programs. In other words, the processing circuitry 223 having read the computer programs has the functions illustrated inside of processing circuitry 223 in FIG. 12. It is also possible to implement the processing circuitry 223 by combining a plurality of independent processors, and by causing each of the processors to execute corresponding one of the computer programs.

The receiving function 223a has the same function as that of the receiving function 123a according to the first embodiment, and receives the age information of the subject P entered by an operator.

The acquiring function 223b acquires an MR image of the subject P from the MRI apparatus 1100. For example, in the synthetic MRI, eight different types of MR images are captured using an imaging process referred to as multi-dynamic multi-echo (MUME) in which two different TEs and four different delay times are combined, per a single run of the imaging.

The acquiring function 223b acquires the eight types of MR images from the MRI apparatus 1100. The acquiring function 223b sends the acquired MR images to the estimating function 223d and the image processing function 223f. The imaging process executed in the MRI apparatus 1100 is not limited to MUME, and the number of MR images to be acquired is not limited to the eight types, either. For example, the acquiring function 223b acquires a plurality of MR images captured by the MRI apparatus 1100 using a pulse sequence including different imaging parameters that are at least related to the magnetization relaxation time.

The identifying function 223c has the same function as that of the identifying function 123b according to the first embodiment, and searches the relaxation characteristic information 1221 for the age finding a match with the age specified in the age information received by the receiving function 223a. The identifying function 223c identifies the T1 value and the T2 value that are mapped to the matching age, as the T1 value and the T2 value of the subject P. The identifying function 223c sends the identified T1 value and T2 value to the estimating function 223d.

The estimating function 223d estimates a magnetization relaxation curve for the subject P, based on the magnetization relaxation characteristic identified by the identifying function 223c, and on the MR images acquired by the acquiring function 223b.

For example, the estimating function 223d estimates the T1 recovery curve and the T2 attenuation curve, based on the signal intensities in the eight types of MR images captured by the synthetic MRI, and the T1 value and the T2 value of the subject P identified by the identifying function 223c.

For example, when the synthetic MRI captures the eight types of MR images described above, measurements of horizontal relaxation time at four points in time, and horizontal relaxation time at two points in time are collected. The estimating function 223d estimates the T1 recovery curve and the T2 attenuation curve of the subject P by adjusting the T1 value and the T2 value of the subject P identified by the identifying function 223c, based on the T1 recovery curve and the T2 attenuation curve having been estimated based on the measurements of the horizontal relaxation time and the horizontal relaxation time. The estimating function 223d sends the estimated T1 recovery curve and the T2 attenuation curve to the determining function 223e.

The determining function 223e determines a plurality of image processing parameters allowing one or more calculated images to be acquired from the MR images acquired by the acquiring function 223b, based on the relaxation curves estimated by the estimating function 223d. The one or more calculated images are those having contrast different from those of the MR images acquired by the acquiring function 223b.

The image processing function 223f generates one or more MR images having different contrast from those of the MR images acquired by the acquiring function 223b from the MR images, using the image processing parameters that are based on the age information received by the receiving function 223a, and on the relaxation characteristic information 1221. The relaxation characteristic information 1221 is stored in the memory 222, for example.

In the present embodiment, a plurality of types of MR images generated by the image processing function 223f are referred to as calculated images.

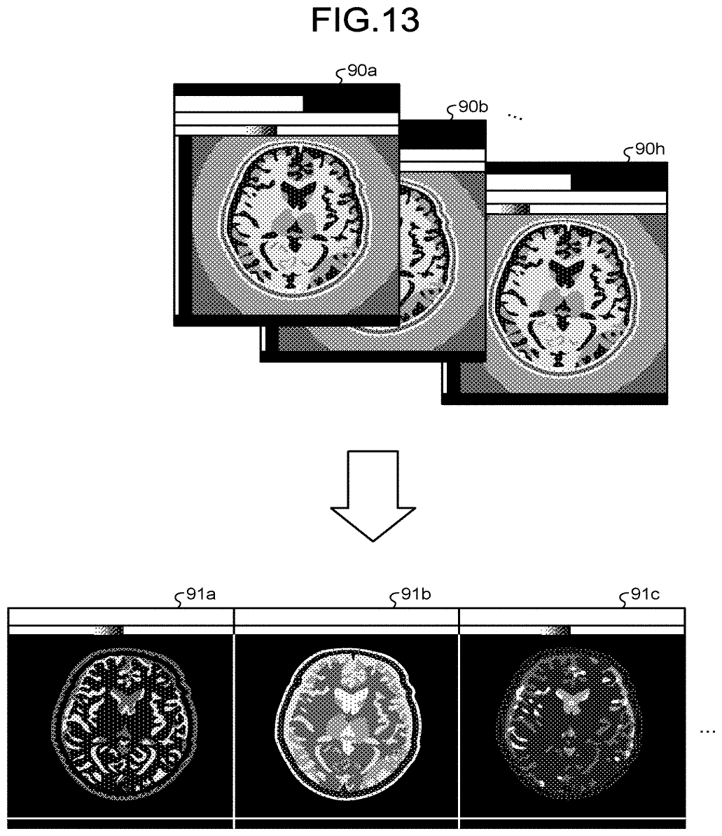

FIG. 13 is a conceptual schematic illustrating one example of MR images 90a to 90h captured by the MRI apparatus 1100, and calculated images 91a to 91c generated by the image processing function 223f according to the embodiment.

More specifically, the image processing function 223f performs image processing for generating the calculated images 91a to 91c from the MR images 90a to 90h acquired by the acquiring function 223b, respectively, using a plurality of image processing parameters determined by the determining function 223e. The calculated images 91a to 91c are equivalent to images acquired by executing a plurality of runs of imaging using different TEs, TRs, or flip angles.

The image processing function 223f generates a plurality of types of calculated images 91a to 91c corresponding to a T1 weighted image, a T2 weighted image, and the like, by performing the image processing. The types and the number of the calculated images are not limited thereto.

Furthermore, the output function 223g displays the calculated images 91a to 91c generated by the image processing function 223f, on the display 225. The output function 223g may also output the calculated images 91a to 91c to another information processing apparatus.

The sequence of the image processing performed by the image processing apparatus 200 having the structure described above will now be explained.

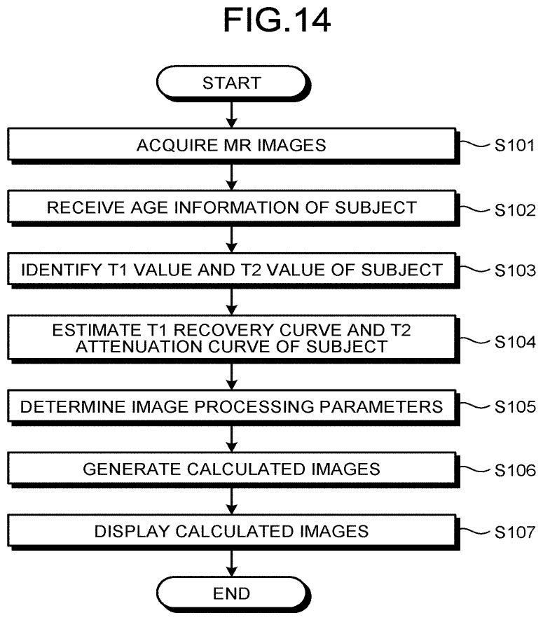

FIG. 14 is a flowchart illustrating one example of the sequence of the image processing according to the embodiment.

To begin with, the acquiring function 223b acquires the MR images 90a to 90h of the subject P from the MRI apparatus 1100 (S101). The acquiring function 223b sends the acquired MR images 90a to 90h to the estimating function 223d and the image processing function 223f.

The receiving function 223a receives the age information of the subject P entered by the operator (S102). The receiving function 123a then sends the received age information to the identifying function 223c.

The identifying function 223c then identifies the T1 value and the T2 value of the subject P based on the age information received by the receiving function 223a, and the relaxation characteristic information 1221 stored in the memory 222 (S103). The identifying function 223c then sends the identified T1 value and T2 value to the estimating function 223d.

The estimating function 223d estimates the T1 recovery curve and the T2 attenuation curve of the subject P from the magnetization relaxation characteristic identified by the identifying function 223c, and from the MR images 90a to 90h acquired by the acquiring function 223b (S104).

The determining function 223e then determines a plurality of image processing parameters based on the T1 recovery curve and the T2 attenuation curve estimated by the estimating function 223d (S105).

The image processing function 223f then generates the calculated images 91a to 91c using a plurality of image processing parameters determined by the determining function 223e (S106).

The output function 223g then displays the calculated images 91a to 91c generated by the image processing function 223f, on the display 225 (S107). The process illustrated in the flowchart is then ended.

Conventionally, when the subject is a child, it has been sometimes difficult to generate a calculated image with appropriate contrast using the image processing parameters for an adult, due to the effects of the development of the child, for example. Therefore, it sometimes takes a long time to perform the image processing, because the operator goes through the trial-and-errors to establish settings of the image processing parameters. Furthermore, when the subject is in the process of growing up, it has been sometimes difficult to use the image processing parameters previously used as they are, and it takes a long time to set the image processing parameters every time an image is to be captured.

By contrast, the image processing apparatus 200 according to the embodiment generates one or more calculated images 91a to 91c using the image processing parameters that are based on the age information of the subject P received from the operator, and on the relaxation characteristic information 1221. Therefore, with the image processing apparatus 200 according to the embodiment, it is possible to reduce the time required in setting the image processing parameters based on the age of the subject P.

Explained in the present embodiment is an example in which the image processing apparatus 200 is provided as an apparatus separate from the MRI apparatus 1100, but the MRI apparatus 1100 may also be provided with the function of the image processing apparatus 200.

Furthermore, explained in the present embodiment is an example in which the image processing function 223f automatically generates the calculated images 91a to 91c based on the image processing parameters determined by the determining function 223e, but the sequence in which the calculated images 91a to 91c are generated is not limited thereto. For example, the output function 223g may display a screen including the image processing parameters determined by the determining function 223e on the display 225, and enable the operator to change the image processing parameters in the screen.

Furthermore, explained in the present embodiment is an example in which the receiving function 223a receives the age information of the subject P entered by the operator, but it is also possible to use a configuration in which the acquiring function 223b acquires the age information of the subject P from the MRI apparatus 1100 or another external apparatus.

Furthermore, the estimating function 223d may adjust the T1 recovery curve and the T2 attenuation curve based on the height, the weight, the sex, the speed of growth, and the like of the subject P, as well as on the age information.

According to at least one of the embodiments explained above, it is possible to reduce the time required in setting the parameters to be used in imaging a magnetic resonance image or in image processing.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic resonance imaging apparatus comprising a processor configured to:
acquire age information representing a length of time elapsed from birth of a subject, and
output a parameter of a pulse sequence for imaging an image of the subject, in a manner suitable for a magnetization relaxation characteristic of the subject, the magnetization relaxation characteristic being based on the acquired age information, and on relaxation characteristic information including mapping of the age information and the magnetization relaxation characteristic.

2. The magnetic resonance imaging apparatus according to claim 1, wherein
the processor is also configured to:
identify the magnetization relaxation characteristic of the subject based on the age information and the relaxation characteristic information;
estimate a magnetization relaxation curve of the subject from the identified magnetization relaxation characteristic;
determine the parameter in a manner suitable for the estimated relaxation curve; and
output the determined parameter.

3. The magnetic resonance imaging apparatus according to claim 1, wherein the age information is information representing a monthly age of the subject.

4. The magnetic resonance imaging apparatus according to claim 1, wherein the age information represents an age of the subject who is in childhood, the age being time elapsed from the birth of the subject to time of imaging.

5. The magnetic resonance imaging apparatus according to claim 1, wherein
the magnetization relaxation characteristic is a T1 value or a T2 value, and
the relaxation characteristic information includes mapping of the age information and the T1 value or the T2 value of the subject who is a child.

6. The magnetic resonance imaging apparatus according to claim 1, wherein the parameter includes at least one of repetition time, echo time, and flip angle.

7. The magnetic resonance imaging apparatus according to claim 1, wherein the parameter includes inversion time.

8. The magnetic resonance imaging apparatus according to claim 1, wherein the parameter includes an imaging parameter of a T2 map.

9. The magnetic resonance imaging apparatus according to claim 2, wherein the processor is configured to adjust the relaxation curve based on at least one of a height, a weight, a sex, and a speed of growth of the subject.

10. The magnetic resonance imaging apparatus according to claim 2, wherein the relaxation curve includes at least one of a T1 recovery curve and a T2 attenuation curve.

11. An image processing apparatus comprising a processor configured to:
acquire age information representing a length of time elapsed from birth of a subject;
acquire a magnetic resonance image of the subject; and
generate one or more calculated images having contrast different from that of the acquired magnetic resonance image, the calculated images being generated from the magnetic resonance image, using an image processing parameter that is based on the acquired age information, and on relaxation characteristic information including mapping of the age information and a magnetization relaxation characteristic.

12. The image processing apparatus according to claim 11, wherein the image processing apparatus is a synthetic magnetic resonance imaging apparatus.

13. The image processing apparatus according to claim 11, wherein the processor is configured to acquire a plurality of magnetic resonance images with a synthetic magnetic resonance imaging apparatus using different settings of an imaging parameter.

14. The image processing apparatus according to claim 11, wherein the processor is configured to:
identify the magnetization relaxation characteristic of the subject based on the age information and the relaxation characteristic information; and
estimate a magnetization relaxation curve of the subject based on signal intensities of a plurality of magnetic resonance images captured with a synthetic magnetic resonance imaging apparatus using different settings of an imaging parameter, and also based on the identified magnetization relaxation characteristic of the subject.

15. The image processing apparatus according to claim 14, wherein
the magnetization relaxation characteristic is a T1 value or a T2 value,
the relaxation characteristic information includes mapping of the age information and the T1 value or the T2 value of the subject who is a child, and
the processor is configured to estimate a T1 recovery curve or a T2 attenuation curve, based on signal intensities of the magnetic resonance images, and the identified T1 value or T2 value of the subject.

16. The image processing apparatus according to claim 14, wherein the processor is configured to determine a plurality of image processing parameters enabling one or more calculated images having contrast different from that of the acquired magnetic resonance images, the calculated images being generated from the magnetic resonance images, the image processing parameters being determined in a manner suitable for the estimated relaxation curve.

17. The image processing apparatus according to claim 11, wherein the age information is information representing a monthly age of the subject.

18. The image processing apparatus according to claim 11, wherein the age information represents an age of the subject who is in childhood, the age being time elapsed from the birth of the subject to time of imaging.

19. The image processing apparatus according to claim 14, wherein the processor is configured to adjust the relaxation curve based on at least one of a height, a weight, a sex, and a speed of growth of the subject.

20. The image processing apparatus according to claim 14, wherein the relaxation curve includes at least one of a T1 recovery curve and a T2 attenuation curve.
